(19) United States
(12) Patent Application Publication
(10) Pub. No.: US 2007/0010454 A1
(43) Pub. Date: Jan. 11, 2007

(54) Method for treating tumor cells resulting in minimal liver toxicity

(76) Inventor: Majid Y. Moridani, Amarillo, TX (US)

Correspondence Address: TEXAS TECH UNIVERSITY;OFFICE OF TECHNOLOGY TRANSFER, P.O. BOX 42007, LUBBOCK, TX, 79409, US

(21) Appl. No.: 11/456,026

(22) Filed: Jul. 6, 2006

Related U.S. Application Data
(60) Provisional application No. 60/697,005, filed on Jul. 6, 2005.

(51) Int. Cl. A61K 38/05; A61K 31/22; A01N 31/08

(52) U.S. Cl. 514/18; 514/546; 514/731

(57) ABSTRACT

A method for treating tumor cells resulting in minimal liver toxicity. An illustrative embodiment includes a method for inhibiting growth of a tumor in a mammal, comprising contacting tumor cells which have tyrosinase activity or P450 activity with 4-t-butoxyphenol, a cytotoxic phenolic composition administered at a dose sufficient to induce tumor cell death with minimal toxicity to the liver. Another illustrative embodiment may be a method for inhibiting growth of a tumor in a mammal, comprising contacting tumor cells which have tyrosinase activity or P450 activity with 4-n-hexyloxyphenol, a cytotoxic phenolic composition administered at a dose sufficient to induce tumor cell death with minimal toxicity to the liver.

### CROSS-REFERENCE TO RELATED APPLICATIONS

This application claims the benefit of provisional application 60/697,005 filed on Jul. 6, 2005.

### STATEMENT REGARDING FEDERALLY SPONSORED RESEARCH OR DEVELOPMENT

Not applicable

### BACKGROUND OF THE INVENTION

Introduction

Malignant melanoma is one of the deadliest cancers known to man. It is estimated that 55,100 new invasive melanoma cases are diagnosed in the USA every year of which 7,910 will be expected to die from the disease. The estimated lifetime risk for melanoma among Americans is 1 in 74. Currently, the therapy for melanoma includes surgical intervention which has a high rate of treatment failure in highly metastatic and advanced cases which are usually fatal. Systemic chemotherapy is often the only resource, but the results to date have been very disappointing and the lack of selective cytotoxicity often leads to intolerable side effects. With increasing occurrence of this disease, there is a clear and urgent need for an improved treatment regimen with enhanced specificity.

Tyrosinase, an enzyme found abundantly only in melanocytes, was selected as a molecular target for 4-hydroxyanisole (4-HA) in the past. 4-HA is a simple phenolic agent which was first shown by Riley to be a melanocytotoxic agent. Tyrosinase was shown to catalyze the oxidation of 4-HA to 4-methoxycatechol and its o-quinone, which reacted readily with nucleophiles. In addition, melanoma toxicity may result from the covalent binding of the o-quinone to protein tiols and/or glutathione (GSH) depletion and inhibition of mitochondrial electron transport. This ultimately leads to desirable melanoma cell death.

4-HA was the only compound from this class that was tested in clinical trials as an anti-melanoma agent. Depigmentation and tumor shrinkage resulted from both the topical application of 4-HA and intra-arterial infusions of 4-HA into patients' legs. Unfortunately, 4-HA clinical trials were terminated because serious liver damage occurred but there were no insights into the mechanisms resulting in induced liver toxicity. It was recently shown that 4-HA was also metabolized by liver P450s via arene epoxidation route to p-quinone (FIG. 1), a reactive metabolite, which was highly toxic to isolated rat hepatocytes.

### SUMMARY OF SOME OF THE PREFERRED EMBODIMENTS

The problems noted above are solved in part by a method for treating tumor cells resulting in minimal liver toxicity. An illustrative embodiment includes a method for inhibiting growth of a tumor in a mammal, comprising contacting tumor cells which have tyrosinase activity or P450 activity with 4-t-butoxyphenol, a cytotoxic phenolic composition administered at a dose sufficient to induce tumor cell death with minimal toxicity to the liver.

Another illustrative embodiment may be a method for inhibiting growth of a tumor in a mammal, comprising contacting tumor cells which have tyrosinase activity or P450 activity with 4-n-hexyloxyphenol, a cytotoxic phenolic composition administered at a dose sufficient to induce tumor cell death with minimal toxicity to the liver.

The tumor cells may be melanoma or hepatoma tumor cells and may originate from a primary tumor or a may be ametastasis from a primary tumor. The tumor cells may be derived from a solid tumor or a non-solid tumor and they may originate from a carcinoma or a sarcoma.

In an embodiment, the composition may be administered orally, parenterally or topically. In some embodiments, conjugates of the composition may be made by conjugating a mono or di glutathione conjugate or a pharmaceutically acceptable ester or salt thereof to a cytotoxic phenolic composition which is also a substrate for tyrosinase or liver P450 enzyme.

The disclosed materials and methods comprise a combination of features and advantages which enable it to overcome the deficiencies of the prior art. The various characteristics described above, as well as other features, will be readily apparent to those skilled in the art upon reading the following detailed description, and by referring to the accompanying drawings.

### BRIEF DESCRIPTION OF THE DRAWINGS

FIG. 1 shows a metabolism pathway in accordance with embodiments of the invention.

FIG. 2 shows a chemical structure of alkoxyphenols in accordance with embodiments of the invention.

FIG. 3 shows cytoxicity of alkoxyphenols in accordance with embodiments of the invention.

FIG. 4 shows a graphical presentation of quantitative structure relationship in accordance with embodiments of the invention.

FIG. 5 shows a metabolism chart in accordance with embodiments of the invention.

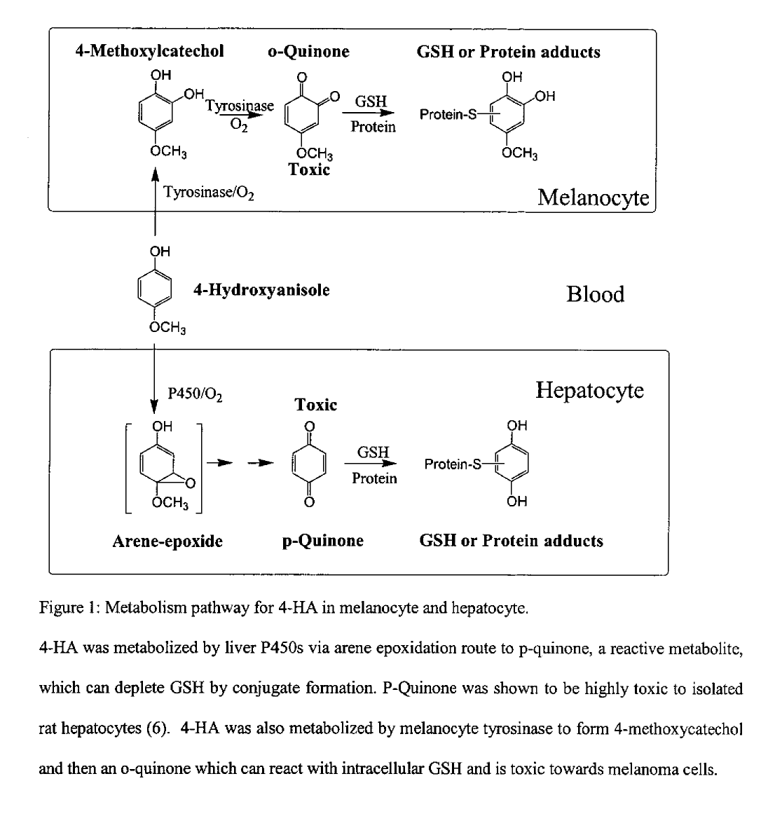

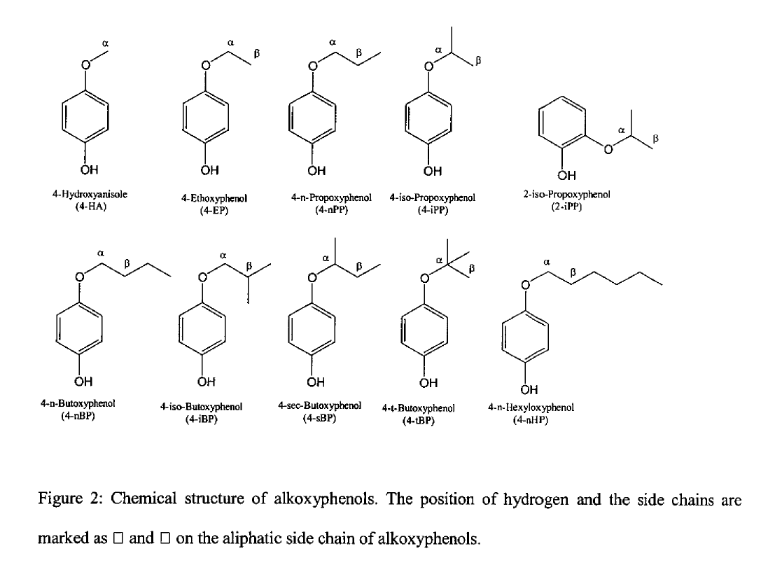

TABLE 1 shows GSH depletion in accordance with embodiments of the invention.

TABLE 2 shows percentage of GSH depletion in accordance with embodiments of the invention.

### DETAILED DESCRIPTION OF THE PREFERRED EMBODIMENTS

The preferred embodiments of the disclosed methods were derived as a result of research performed to identify a phenolic compound with minimum toxicity towards the liver but yet efficacious against melanoma. Ten alkoxyphenol compounds with various linear aliphatic side chains and their branched analogues were investigated for their metabolism by tyrosinase/O₂, rat liver P450 microsomal preparation/NADPH/O₂ metabolizing systems, and for their toxicity towards the B16-F0 mouse melanoma cell line. The data indicated that all alkoxyphenols tested in this work demonstrated toxicity towards murine B16-F0 melanoma cell line. 4-nHP (4-n-hexyloxyphenol) demonstrated a significant advantage over other alkoxyphenols with respect to GSH depletion by rat liver microsomal P450s and therefore its toxicity towards the liver.

In accordance with embodiments of the disclosed methods, tyrosinase, an enzyme present abundantly in melanocytes was selected as a molecular target for the treatment of malignant melanoma. Ten alkoxyphenols were investigated for their metabolism by tyrosinase/O₂, rat liver P450 microsomal/NADPH/O₂ metabolizing systems and for their toxicity towards B16-F0 melanoma cells.

All ten alkoxyphenols showed a dose- and time-dependent toxicity towards B16-F0 cells except 2-iso-propoxyphenol. 4-n-Hexyloxyphenol demonstrated the greatest toxicity towards B16-F0 cells while minimally depleting glutathione in microsomal preparations at its calculated LC₁₀ and LC₅₀ lethal concentrations for B16-F0. At 100 □M concentrations, 4-t-butoxyphenol showed the lowest amount of glutathione depletion by microsomal P450 system. Alkoxyphenols with at least two alkyl groups derivatized at alpha carbon of alkoxy group showed minimal rates of metabolism by tyrosinase/O₂ metabolizing system. A quantitative structural toxicity relationship equation was also derived, LogLC₅₀(□M)=−0.265(±0.064)LogP+2.482(±0.179).

4-n-hexyloxyphenol was identified as a potential lead anti-melanoma agent against B16-F0 melanoma cells with minimal metabolism by liver P450 microsomal preparation.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference in the Description of Related Art is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

### EXAMPLES

The invention having been generally described, the following examples are given as particular embodiments of the invention and to demonstrate the practice and advantages hereof. It is understood that the examples are given by way of illustration and are not intended to limit the specification or the claims to follow in any manner.

### Example 1

UV-VIS spectroscopy of tyrosinase mediated metabolism of alkoxyphenols The spectra of a solution containing alkoxyphenol (100 □M) and tyrosinase (20 U/mL) were recorded in the absence and presence of GSH (200 □M) using a GBC UV-Visible spectral spectrophotometer (GC Scientific, Australia). The spectra of the mixture were obtained when GSH was added to the solution either before or after the addition of tyrosinase. The control spectrum was that of the respective alkoxyphenol solution (100 □M) in phosphate buffer [0.1 M (pH 7.4) containing DETAPAC (1 mM)].

Tyrosinase Mediated GSH Depletion Assay

Tyrosinase (10 □L; 2500 U/mL) was added to a mixture of alkoxyphenol (100 □M) and GSH (200 □M) in a final volume of 1 mL phosphate buffer (0.1 M, pH 7.4, DETAPAC 1 mM). The mixture was pre-incubated for 30, 90, and 180 min at 37° C. A 250 □L aliquot was added to trichloroacetic acid (25 □L; 30% w/v), vortexed and left at room temperature for 5 min. A 100 □L aliquot of the supernatant was then added to a mixture of Ellman's reagent 5,5′-dithiobis-(2-nitrobenzoic acid) DTNB (25 □L; 2 mg/mL) and Tris/HCl buffer (875 □L; 0.1 M, pH 8.94), and then vortexed. The absorbance of the solution was observed at 412 nm (12, 13). The standard curve for GSH measurement gave a regression coefficient of greater than 0.99 over the range of 5-200 □M GSH concentrations (data not shown).

Animal Housing and Protocol

Adult male Sprague-Dawley rats, 250-300 g, were obtained from Charles River Laboratories, USA, fed ad libitum, were allowed to acclimatize for 1 week on clay chip bedding in a room with a 12 h light photocycle, an environmental temperature of 21-23° C. and 50-60% relative humidity.

Rat Liver Microsomal Preparation

The rats were anesthetized by sodium pentobarbital (60 mg/kg) before surgery in order to prepare the animal before liver removal. Hepatic microsomes were prepared by differential centrifugation as described previously (14). Briefly, the liver was removed and weighed in a beaker on ice. The liver was cut into pieces and washed by cold KCl (154 mM): Tris/HCl (50 mM, pH 7.4) buffer solution then suspended into 4 volumes of KCl:Tris/HCl buffer. The tissue was gently homogenized using an electrical homogenizer and subsequently by a handheld glass tissue grinder before centrifuging at 1935 g (Beckman Avanti J-251, Beckman Rotor-JA-25.5) at 4° C. for 15 mm to remove tissue and cell debris. The supernatant was centrifuged at 12,100 g at 4° C. for 15 min to remove subcellular organelles followed by centrifigation at 100,000 g (Beckman Optima LE-80K, Beckman Rotor-45 Ti) at 4° C. for 1 h. The supernatant was discarded and micorsomes were separated and suspended in 5 mL Tris/HCl buffer (100 mM, pH 7.4) containing 1 mM DETAPAC. The mixture was homogenized using a handheld glass tissue grinder after which an additional 15 mL Tris HCl buffer was added and the solution was aliquoted in 750 □L and stored at −70° C. for subsequent use. Microsomal protein content was determined by a modified Lowry method (15).

CYP2E1 Induced Rat Liver Microsomal Preparation

CYP2E1 induced microsomes were prepared from rats treated (i.p.) with inducing agent pyrazole (200 mg/kg/day) (16) for 2 consecutive days before sacrificing the rats on the 3^{rd} day.

Microsomal Mediated GSH Depletion Assay

The amount of GSH conjugates formed were determined colorimetrically using Ellman's reagent 5,5′-dithiobis-(2-nitrobenzoic acid) (DTNB) (13). Incubation mixtures contained in a final volume of 1 mL phosphate buffer (0.1 M, pH 7.4, DETAPAC 1 mM), 2 mg/mL rat liver microsomes, 200 □M GSH, 1 mM NADPH, and 100 □M alkoxyphenols. The mixtures were gently mixed at 37° C. from which 250 □L aliquots were taken at 30, 60, and 90 min time-interval points into Eppendorf tubes containing 25 □L trichloroacetic acid (30% w/v). Following protein precipitation and centrifugation for 5 min, the GSH levels of a 100 □L aliquot of the supernatant was determined by the addition of 0.1 M Tris/HCl buffer, pH 8.94 (875 □L), and 2 mg/mL DTNB (25 □L). The reduced DTNB formed was determined at 412 nm on a GBC spectral spectrophotometer.

Cell Culture

The mouse B16-F0 cell line was obtained from American Type Culture Collection, USA. A frozen B16-F0 cell vial was washed twice with DMEM media before culturing in 8 mL DMEM supplemented with fetal calf serum (FCS) (10%) and streptomycin/penicillin (100 U/mL) in a T-25 flask (17). All the cell culture processes were carried out in a type II vertical laminar air flow chamber. The B16-F0 cultures were kept at 37° C. under a 5% CO₂ atmosphere in a TS Autoflow CO₂ Water-Jacketed Incubator (NUAIRE, USA). The culture medium was changed when acidification was indicated by the pH indicator and when needed. To detach the cells from the flask, the media was first removed from the flask. The remaining media was then washed out using sterile phosphate buffered saline (PBS) (2-3 mL). Subsequently, Versene (2-5 mL) was added to the flask and the sample was incubated for 2-3 min in the incubator to provide time for cells to detach. The detached cells were rinsed with ˜10 mL of pre-warmed sterile PBS to dilute Versene. The mixture was transferred into a 50 mL tube. The flask was additionally rinsed with sterile PBS and the content was added to the rest of the cells collected. The cells were then spun down at 800 RPM (Beckman GPR Centrifuge, USA) for 3-5 min. The PBS-Versene mixture was aspirated off. Pelletted cells were then re-suspended in DMEM media (supplemented by FCS 10% and antibiotics 100 U/mL) followed by splitting the mixture into one T-75 flask containing 30 mL media (25% of the media was supplemented from the previous culture step as conditioning media).

Cell Viability

To determine cell viability, the cells obtained from each flask were suspended in 4 mL of DMEM media supplemented by FCS 10% and antibiotics 100 U/mL (contained 25% media from the previous culture step as conditioning media). The cells were counted using trypan blue exclusion method (18) for determining the viability.

MTT Assay in 96 Well Plates

To evaluate cytotoxicity, cells were obtained from exponentially growing 90-95% confluent cultures and seeded at 12,500 cells/well in 96-well plates. The cells were kept in 100 □L fresh DMEM media (supplemented by FCS 10% and antibiotics 100 U/mL) for 24 h to allow cell adhesion and environmental adaptation. Subsequently, the cells were treated with additional 150 □L DMEM (supplemented with FCS 10% and antibiotics 100 U/mL) containing various concentrations of alkoxyphenols for 1-4 days. At 24 h interval, the medium was removed and the wells were washed three times using DMEM media alone before adding 40 □L of 2 mg/mL yellow tetrazolium dye (3-(4,5-dimethylthiazolyl-2)-2,5-diphenyltetrazolium bromide) (MTT) (17). The plates were returned to the incubator for a period of 4 h. The residual MTT solutions were removed from wells and then 200 □L of DMSO was added to each well. The plates were stored at room temperature in a dark place for an additional 2 h before reading them at 570 nm using XFluor Plate Reader (Tecan US, Inc, USA).

All experiments were performed in triplicate. An analysis of variance (ANOVA) of repeated means was carried out to compare the percentage of surviving cells in the cultures for different concentrations of each compound. t-test was used to compare the results of toxicity of the alkoxyphenols with 4-HA.

LC₁₀ and LC₅₀ Calculation

Lethal concentrations (LC) which can cause 10% and 50% of the cell death were calculated from the linear regression equation derived from graphing the viability of the cells at day 2 (on x axis) versus the concentration of the drug (on y axis).

Partition Coefficient

Partition coefficient values were estimated using the LogP software available at www.LogP.com.

UV-VIS spectroscopy of tyrosinase mediated metabolism of alkoxyphenols The progression of alkoxyphenol's oxidation was monitored by tyrosinase/O₂-oxidizing system using a UV-VIS spectroscopy method which showed a distinct peak at 420-470 nm with a characteristic indicative of o-quinone formation. Addition of glutathione at the beginning of the metabolism reaction resulted in the significant loss in the absorbance of the 420-470 nm peaks. The UV-VIS spectra of this peak for all the alkoxyphenols were developed over 1 min except for 2-iPP and 4-tBP which were found to have a 20-fold lower rate of oxidation by tyrosinase/O₂ metabolizing system.

Tyrosinase Mediated GSH Depletion Assay

As shown in Table 1, alkoxyphenols depleted GSH in the tyrosinse/O₂ metabolizing system in the following decreasing order: 4-HA>>4-nBP, 4-iBP, and 4-nHP>>4-EP and 4-nPP>>4-iPP, 2-iPP, 4-sBP and 4-tBP. 4-HA depleted 1.8 molecular equivalent of GSH per mole at 30 min incubation. 4-nBP, 4-iBP, and 4nHP depleted 1.8 molecular equivalent of GSH per mole whereas 4-EP and 4-nPP depleted GSH on mole per mole basis after 90 min incubation. 4-iPP, 2-iPP, 4-sBP and 4-tBP depleted GSH only to an average equivalent of 0.15 molar per mole with tyrosinase/O₂ metabolizing system during the 90 min incubation. It was found that additional substitution/s on the alpha carbon of alkoxy group prevented the metabolism of the molecule by tyrosinase/O₂. For instance, the rate of metabolism for 4-butoxyphenol series (4-nBP, 4-iBP, 4-sBP, and 4-tBP) by tyrosinase was diminished in the following decreasing order 4-tBP<4-sBP<4-nBP and 4-iBP<4-HA which suggests that the presence of a non linear side chain on the alpha carbon atom of the alkoxy group may interfere with the molecular fit into the tyrosinase enzyme active site. Negligible GSH depletion occurred in the absence of the enzyme.

Glutathione Conjugate Formation by Rat Hepatocyte Microsomes

The amount of GSH depleted as a result of alkoxyphenol metabolism catalyzed by rat liver microsomes/NADPH/O₂ was determined to be between 1.2-1.5 equivalents of GSH per mole except for 4-tBP which was 0.5 equivalent per mole of GSH after 90 min of incubation. At 100 □M concentration (Table 1), 4-tBP showed the lowest rate of GSH depletion by microsomal P450/NADPH/O₂ metabolizing system which corresponded to 2.3 fold less than 4-HA. This suggests that 100% of the alkoxyphenols in the reaction mixture underwent glutathione conjugation except 4-tBP which could be due to hindrance imposed by the presence of the bulky t-butoxy group in its molecular structure. It was noted that the alkoxyphenols were metabolized by CYP 2E1 induced rat liver microsomes more readily than when non-induced/standard rat microsomal P450 system was used. Negligible GSH depletion occurred in the absence of the enzyme.

Cell Viability

Cell viability of the cultured murine B16-F0 melanoma cell line was measured using trypan blue exclusion test (18) and was always greater than 95% before seeding the cells into the 96 well plates for MTT assay.

Cytotoxicity in B16-F0 Melanoma Cell Line

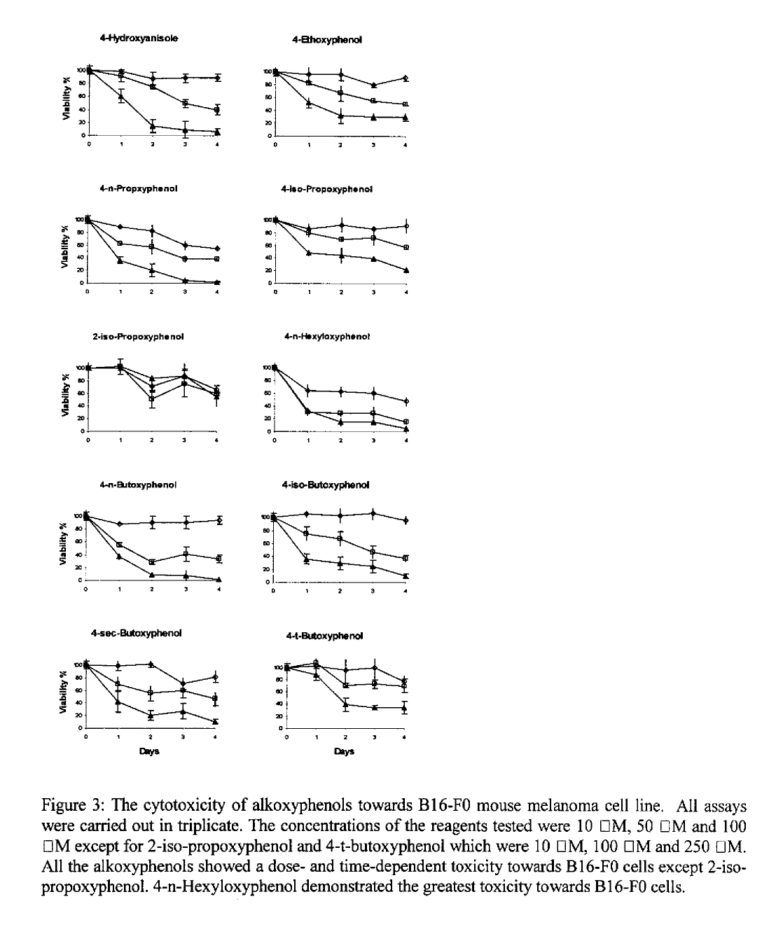

The LC₅₀ (2 day) concentrations were determined by MTT assay (17) as a measure of melanoma cell viability (FIG. 3). The required concentration of compounds that can cause 50% decrease in melanoma cell viability (LC₅₀ □M) on the second day are given in Table 1. ANOVA and regression analysis of the toxicity of each of the alkoxyphenols at various doses showed the cytotoxicity to be dose- and time-dependent with a ranking order of 4-nHP>>4-nPP, 4-HA, 4-sBP, 4-iBP, 4-EP>4-iPP>>4-tBP>2-iPP except for 2-iPP.

Partition Coefficient

The partition coefficients of the alkoxyphenols were estimated using the LogP software (www.logp.com) and were in a decreasing order of 4-nHP>>4-nBP, 4-iBP, 4-s-BP, 4-tBP>>4-nPP>4-1-PP and 2-iPP>>4-EP>>4-HA, thus indicating that the lipid solubility of the alkoxyphenol increases as the size of the aliphatic group and/or the number of carbon atoms on the aliphatic side chain group increases (Table 1).

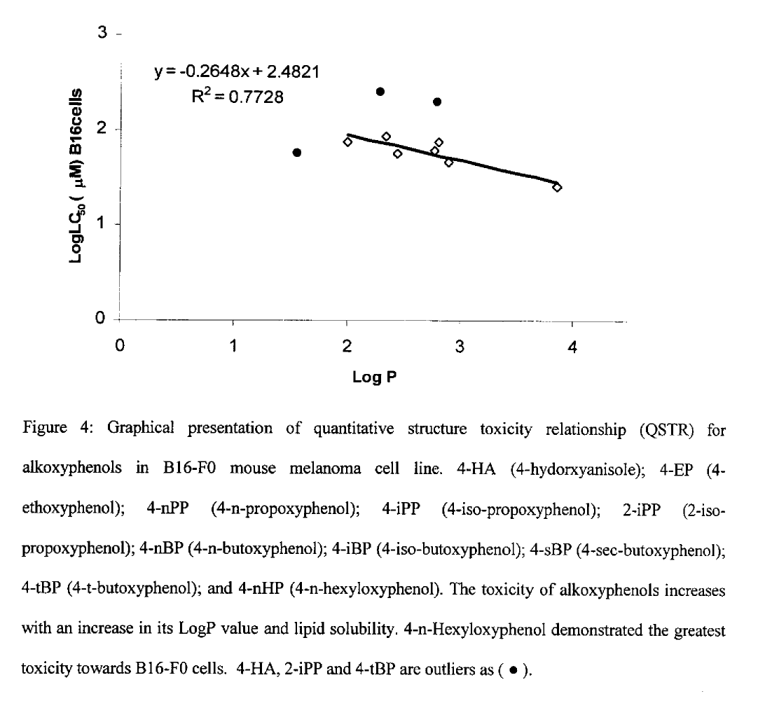

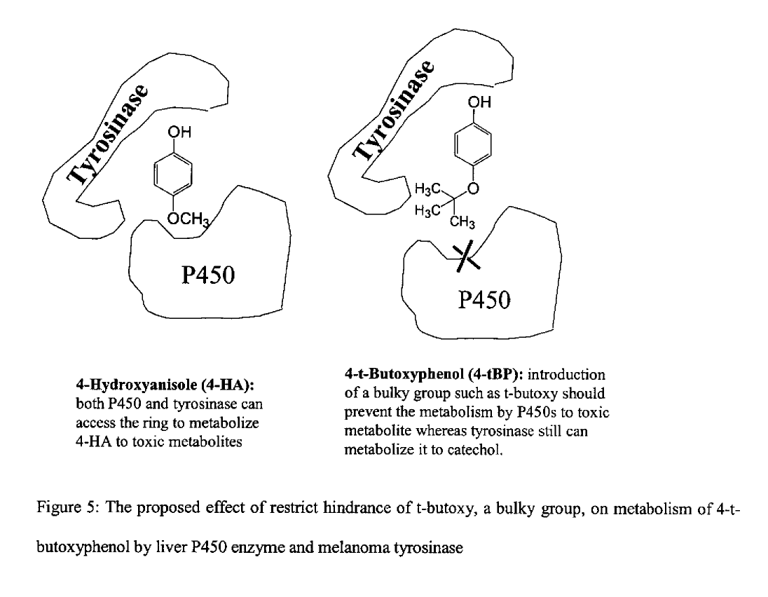

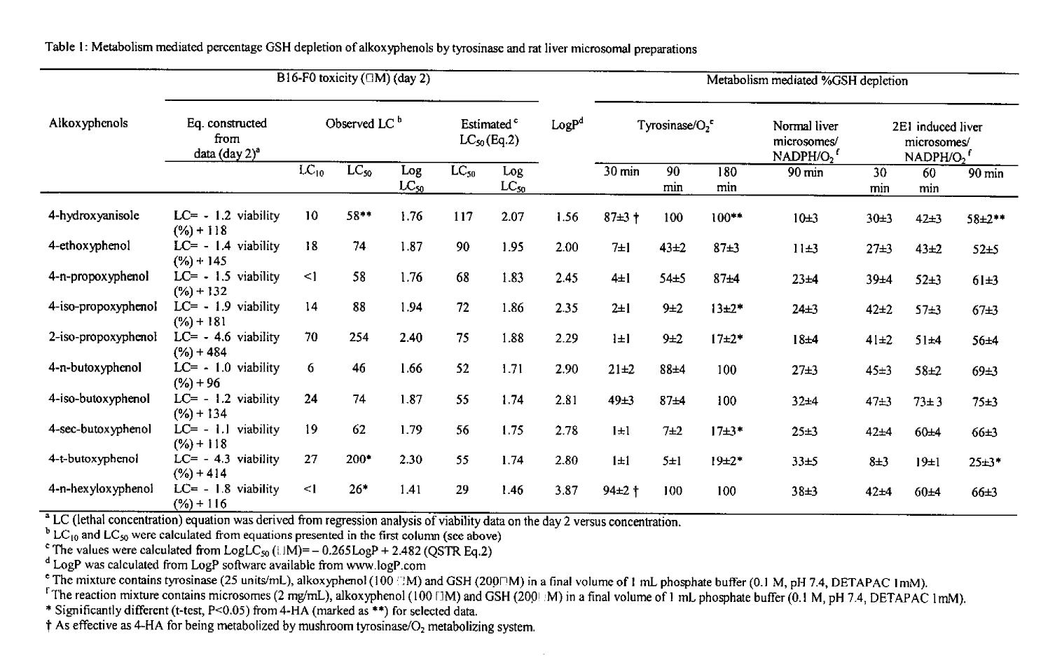

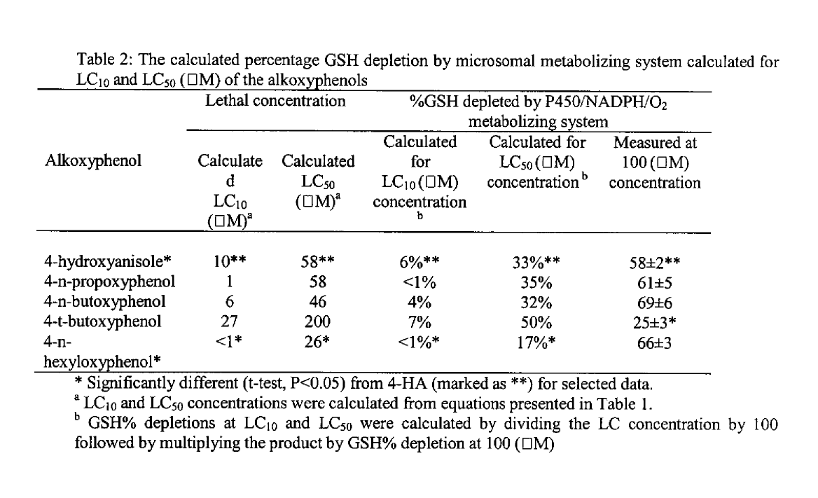

One parameter quantitative structure toxicity relationship The data in Table 1 was used to derive Eq. 1 as a one-parameter model for quantitative structure toxicity relationship (QSTR) for describing the toxicity of the alkoxyphenols towards B16-F0 melanoma cell line.
LogLC₅₀ (□M)=−0.182 (±0.153) LogP+2.345 (±0.405) Eq. 1
(n=10, R²=0.150, P value for LogP term=0.268; P value for intercept term<0.001)
As shown in Eq. 2 (FIG. 4) the exclusion of outliers (4-HA, 2-iPP and 4-tBP; shown as ≡ symbol) from the rest of the data points (shown as ⋄ symbol) greatly improved the QSTR equation between the alkoxyphenols LogLC₅₀ (□M) and their LogP values. The calculated LogLC₅₀ values from Eq. 2 were similar to the experimental data (Table 1). The outlier 4-HA (Table 1) was 1.7 fold more toxic than the toxic value calculated from Eq. 2 whereas both 2-iPP and 4-tBP were 3.6 fold less toxic. However the toxicity of the seven alkoxyphenols was well predicted by Eq. 2.
LogLC₅₀ (□M)=−0.265 (±0.064) LogP+2.482 (±0.179) Eq. 2
(n=7, R²=0.773, P value for LogP term=0.009; P value for intercept term<0.0001)
Outliers: 4-HA, 2-iPP, and 4-tBP

What is claimed is:

1. A method for inhibiting growth of a tumor in a mammal, comprising contacting tumor cells which have tyrosinase activity or P450 activity with 4-t-butoxyphenol, a cytotoxic phenolic composition administered at a dose sufficient to induce tumor cell death with minimal toxicity to the liver.

2. The method according to claim 1 wherein said tumor cells may be melanoma tumor cells.

3. The method according to claim 1 wherein said tumor cells may be hepatoma tumor cells.

4. The method according to claim 1, wherein said tumor cells may be a primary tumor.

5. The method according to claim 1, wherein said tumor cells may be a metastasis from a primary tumor.

6. The method according to claim 1, wherein said tumor cells may be derived from a solid tumor or a non-solid tumor.

7. The method according to claim 1, wherein said tumor cells may be a carcinoma or a sarcoma.

8. The method according to claim 1 wherein the composition may be administered orally, parenterally or topically.

9. The method according to claim 1 wherein conjugates of said composition may be made by conjugating a mono or di glutathione conjugate or a pharmaceutically acceptable ester or salt thereof to a cytotoxic phenolic composition which is also a substrate for tyrosinase or liver P450 enzyme.

10. A method for inhibiting growth of a tumor in a mammal, comprising contacting tumor cells which have tyrosinase activity or P450 activity with 4-n-hexyloxyphenol, a cytotoxic phenolic composition administered at a dose sufficient to induce tumor cell death with minimal toxicity to the liver.

11. The method of claim 2 wherein said tumor cells may be melanoma tumor cells.

12. The method of claim 2 wherein said tumor cells may be hepatoma tumor cells.

13. The method according to claim 1, wherein said tumor cells may be a primary tumor.

14. The method according to claim 1, wherein said tumor cells may be a metastasis from a primary tumor.

15. The method of claim 2, wherein said tumor cells may be derived from a solid tumor or a non-solid tumor.

16. The method of claim 2, wherein said tumor cells may be a carcinoma or a sarcoma.

17. The method according to claim 2 wherein the composition may be administered orally, parenterally or topically.

18. The method according to claim 2 wherein conjugates of said cytotoxic phenolic compositions may be made by conjugating a mono or di glutathione conjugate or a pharmaceutically acceptable ester or salt thereof to a cytotoxic phenolic composition which is also a substrate for tyrosinase or liver P450 enzyme.
